(19) United States
(12) Patent Application Publication
(10) Pub. No.: US 2024/0194341 A1
(43) Pub. Date: Jun. 13, 2024

(54) DETERMINING USER-SPECIFIC HYPERPARAMETERS FOR DECISION SUPPORT MODELS

(71) Applicant: Dexcom, Inc., San Diego, CA (US)

(72) Inventors: Joost Herman VAN DER LINDEN, Afton, MN (US); Mark DERDZINSKI, Aliquippa, PA (US); Margaret A. CRAWFORD, San Diego, CA (US); Giada ACCIAROLI, San Diego, CA (US); Christopher R. HANNEMANN, San Diego, CA (US)

(21) Appl. No.: 18/503,486

(22) Filed: Nov. 7, 2023

Related U.S. Application Data
(60) Provisional application No. 63/386,352, filed on Dec. 7, 2022.

(51) Int. Cl. G16H 50/20; G06N 3/0985

(52) U.S. Cl. CPC G16H 50/20; G06N 3/0985

(57) ABSTRACT

Systems, devices, and methods for determining user-specific hyperparameters for decision support models are provided. In one embodiment, a non-transitory computer readable storage medium storing a program is provided, the program comprising instructions that, when executed by at least one processor of a computing device, cause the at least one processor to perform operations including performing an initial exploration phase; performing a training phase; and performing an exploration-exploitation phase by: dividing users into an exploration subset and an exploitation subset; determining at least one optimal hyperparameter for each user of the exploitation subset; determining, using the at least one optimal hyperparameter, at least one decision support output for each user of the exploitation subset; randomly assigning at least one hyperparameter to each user of the exploration subset; and determining, using the at least one randomly assigned hyperparameter, at least one decision support output for each user of the exploration subset.

### CROSS-REFERENCE TO RELATED APPLICATIONS

This application claims benefit of and priority to U.S. Provisional Application No. 63/386,352, filed Dec. 7, 2022, which is assigned to the assignee hereof and hereby expressly incorporated herein in its entirety as if fully set forth below and for all applicable purposes.

### BACKGROUND

### Field of Disclosure

This application generally relates to medical devices (e.g., analyte sensors), and more specifically to systems, devices, and methods for determining hyperparameters for improving patients' health outcomes.

### Description of the Related Technology

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps may also be utilized to receive insulin. Diet and exercise also affect blood glucose levels.

Diabetes conditions may be referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### BRIEF SUMMARY

The various embodiments of the present systems, devices, and methods for determining user-specific hyperparameters for decision support models for improving patients' health outcomes comprise several features, no single one of which is solely responsible for their desirable attributes. Without limiting the scope of the present embodiments, their more prominent features will now be discussed below. After considering this discussion, and particularly after reading the section entitled “Detailed Description,” one will understand how the features of the present embodiments provide the advantages described here.

In a first aspect, a non-transitory computer readable storage medium is provided, the non-transitory computer readable storage medium storing a program comprising instructions that, when executed by at least one processor of a computing device, cause the at least one processor to perform operations including: performing an initial exploration phase by: randomly assigning at least one hyperparameter to each user of a plurality of users; performing a training phase by: analyzing training data, wherein the training data comprises contextual data and a value associated with the at least one randomly assigned hyperparameter; and determining a relationship between the contextual data and the value associated with the at least one randomly assigned hyperparameter; and performing an exploration-exploitation phase by: dividing the plurality of users into an exploration subset of users and an exploitation subset of users; determining at least one optimal hyperparameter for each user of the exploitation subset of users; determining, using the at least one optimal hyperparameter, at least one decision support output for each user of the exploitation subset of users; randomly assigning at least one hyperparameter to each user of the exploration subset of users; and determining, using the at least one randomly assigned hyperparameter, at least one decision support output for each user of the exploration subset of users.

In an embodiment of the first aspect, the initial exploration phase is further performed by: determining, using the at least one randomly assigned hyperparameter, a decision support output for each user of the plurality of users; providing the decision support output to each user of the plurality of users; and receiving monitoring data for each user of the plurality of user, wherein the monitoring data provides information to at least one physiological condition.

In another embodiment of the first aspect, the training data further comprises the monitoring data, and the training phase is further performed by determining a relationship between the monitoring data, the contextual data, and the value associated with the at least one randomly assigned hyperparameter.

In another embodiment of the first aspect, the at least one optimal hyperparameter for each user of the exploitation subset of users is determined by: assigning a plurality of experimental hyperparameters to each user of the exploitation subset; determining at least one predicted outcome for each experimental hyperparameter of the plurality of experimental hyperparameters; determining a scalarized outcome for each experimental hyperparameter of the plurality of experimental hyperparameters; and determining the at least one optimal hyperparameter based on the scalarized outcome.

In another embodiment of the first aspect, the operations further comprise: performing a retraining phase by: analyzing training data, wherein the training data comprises contextual data, a value associated with the randomly assigned hyperparameter of the exploration subset of users, and monitoring data for the exploration subset of users; and determining a relationship between the contextual data, the value associated with the at least one randomly assigned hyperparameter of the exploration subset of users, and the monitoring data for the exploration subset of users.

In another embodiment of the first aspect, the exploration-exploitation phase is performed using a contextual multi-armed bandit algorithm.

In another embodiment of the first aspect, the at least one predicted outcome is determined using a tactic assignment algorithm.

In a second aspect, a method for determining user-specific hyperparameters for decision support models is provided, the method comprising: performing an initial exploration phase by: randomly assigning at least one hyperparameter to each user of a plurality of users; performing a training phase by: analyzing training data, wherein the training data comprises contextual data and a value associated with the at least one randomly assigned hyperparameter; and determining a relationship between the contextual data and the value associated with the at least one randomly assigned hyperparameter; and performing an exploration-exploitation phase by: dividing the plurality of users into an exploration subset of users and an exploitation subset of users; determining at least one optimal hyperparameter for each user of the exploitation subset of users; determining, using the at least one optimal hyperparameter, at least one decision support output for each user of the exploitation subset of users; randomly assigning at least one hyperparameter to each user of the exploration subset of users; and determining, using the at least one randomly assigned hyperparameter, at least one decision support output for each user of the exploration subset of users.

In an embodiment of the second aspect, the initial exploration phase is further performed by: determining, using the at least one randomly assigned hyperparameter, a decision support output for each user of the plurality of users; providing the decision support output to each user of the plurality of users; and receiving monitoring data for each user of the plurality of user, wherein the monitoring data provides information to at least one physiological condition.

In another embodiment of the second aspect, the training data further comprises the monitoring data, and the training phase is further performed by determining a relationship between the monitoring data, the contextual data, and the value associated with the at least one randomly assigned hyperparameter.

In another embodiment of the second aspect, the at least one optimal hyperparameter for each user of the exploitation subset of users is determined by: assigning a plurality of experimental hyperparameters to each user of the exploitation subset; determining at least one predicted outcome for each experimental hyperparameter of the plurality of experimental hyperparameters; determining a scalarized outcome for each experimental hyperparameter of the plurality of experimental hyperparameters; and determining the at least one optimal hyperparameter based on the scalarized outcome.

In another embodiment of the second aspect, the method further comprises: performing a retraining phase by: analyzing training data, wherein the training data comprises contextual data, a value associated with the randomly assigned hyperparameter of the exploration subset of users, and monitoring data for the exploration subset of users; and determining a relationship between the contextual data, the value associated with the at least one randomly assigned hyperparameter of the exploration subset of users, and the monitoring data for the exploration subset of users.

In another embodiment of the second aspect, the exploration-exploitation phase is performed using a contextual multi-armed bandit algorithm.

In another embodiment of the second aspect, the training phase is performed using a tactic assignment algorithm.

In a third aspect, a computing device for determining user-specific hyperparameters for decision support models is provided, the computing device comprising: a network interface; a processor operatively connected to the network interface; a memory storing a program comprising instructions that, when executed by the processor, cause the computing device to: perform an initial exploration phase by: randomly assigning at least one hyperparameter to each user of a plurality of users; perform a training phase by: analyzing training data, wherein the training data comprises contextual data and a value associated with the at least one randomly assigned hyperparameter; and determining a relationship between the contextual data and the value associated with the at least one randomly assigned hyperparameter; and perform an exploration-exploitation phase by: dividing the plurality of users into an exploration subset of users and an exploitation subset of users; determining at least one optimal hyperparameter for each user of the exploitation subset of users; determining, using the at least one optimal hyperparameter, at least one decision support output for each user of the exploitation subset of users; randomly assigning at least one hyperparameter to each user of the exploration subset of users; and determining, using the at least one randomly assigned hyperparameter, at least one decision support output for each user of the exploration subset of users.

In an embodiment of the third aspect, the initial exploration phase is further performed by: determining, using the at least one randomly assigned hyperparameter, a decision support output for each user of the plurality of users using a decision support model; providing the decision support output to each user of the plurality of users; and receiving monitoring data for each user of the plurality of user, wherein the monitoring data provides information to at least one physiological condition.

In another embodiment of the third aspect, the training data further comprises the monitoring data, and the training phase is further performed by determining a relationship between the monitoring data, the contextual data, and the value associated with the at least one randomly assigned hyperparameter.

In another embodiment of the third aspect, the at least one optimal hyperparameter for each user of the exploitation subset of users is determined by: assigning a plurality of experimental hyperparameters to each user of the exploitation subset; determining at least one predicted outcome for each experimental hyperparameter of the plurality of experimental hyperparameters; determining a scalarized outcome for each experimental hyperparameter of the plurality of experimental hyperparameters; and determining the at least one optimal hyperparameter based on the scalarized outcome.

In another embodiment of the third aspect, the computing device is further configured to: perform a retraining phase by: analyzing training data, wherein the training data comprises contextual data, a value associated with the randomly assigned hyperparameter of the exploration subset of users, and monitoring data of the exploration subset of users; and determining a relationship between the contextual data, the value associated with the at least one randomly assigned hyperparameter of the exploration subset of users, and the monitoring data of the exploration subset of users.

In another embodiment of the third aspect, the exploration-exploitation phase is performed using a contextual multi-armed bandit algorithm.

### BRIEF DESCRIPTION OF THE DRAWINGS

FIG. 1A illustrates an example health monitoring and support system, in accordance with certain embodiments of the disclosure.

FIG. 1B illustrates a continuous analyte monitoring system, in accordance with certain embodiments of the disclosure.

FIG. 2 illustrates example inputs and example metrics that are generated based on the inputs, in accordance with certain embodiments of the disclosure.

FIG. 3 is a flow diagram illustrating a process for determining decision support outputs based on user-specific hyperparameters, in accordance with certain embodiments of the disclosure.

FIG. 4 is a flow diagram illustrating a process for performing the initial exploration phase of the process of FIG. 3, in accordance with certain embodiments of the disclosure.

FIG. 5 is a flow diagram illustrating a process for performing the training phase of the process of FIG. 3, in accordance with certain embodiments of the disclosure.

FIG. 6 is a flow diagram illustrating a process for performing the exploration-exploitation phase of the process of FIG. 3, in accordance with certain embodiments of the disclosure.

FIG. 7 is a flow diagram illustrating a process for determining decision support outputs for an exploitation subset of users, in accordance with certain embodiments of the disclosure.

FIG. 8 is a flow diagram illustrating a process for determining decision support outputs for an exploration subset of users, in accordance with certain embodiments of the disclosure.

FIG. 9 is a flow diagram illustrating an exemplary operational process for determining decision support outputs based on user-specific hyperparameters, in accordance with certain embodiments of the disclosure.

FIG. 10 is a block diagram depicting a computing device configured to determine user-specific hyperparameters for use with decision support models, in accordance with certain embodiments of the disclosure.

### DETAILED DESCRIPTION

Portable and/or wearable health monitoring devices (also referred to herein as “health monitoring devices”) and mobile health applications (also referred to herein as “applications”), have rapidly become renowned for their capabilities to support user-centered care. For example, management of diabetes can present complex challenges for patients, clinicians, and caregivers, as a confluence of many factors can impact a patient's glucose level and glucose trends. To assist patients with better managing this condition, health monitoring devices (e.g., sensors and other types of monitoring and diagnostic devices) as well as a variety of mobile health applications (e.g., diabetes intervention software applications) have been developed. The wide dissemination of health monitoring devices and the increase in the development and distribution of mobile health applications has improved health management, and more specifically chronic disease management, in the healthcare domain. In particular, the use of mobile health applications in conjunction with these health monitoring devices, represents a more scalable and potentially more cost effective alternative to traditional interventions, offering a means of improving health and chronic disease management by expanding the reach of healthcare services and improving users' access to health-related information and interventions.

Mobile health applications enable users to be much more involved in the users' own medical care by granting them access to and control over their health information. In particular, mobile health applications enable users to access, monitor, record, and update their health information regardless of physical constraints, such as time and location. In particular, a variety of intervention applications have been developed to deliver guidance that may assist patients, caregivers, healthcare providers, or other users in improving lifestyle or clinical/patient outcomes by meeting a variety of challenges, such as analyte control, exercise, and/or other health factors. The term “analyte” as used herein refers without limitation to a substance or chemical constituent in the body or a biological sample. For example, diabetes intervention applications may assist patients, caregivers, healthcare providers, or other users in overnight glucose control (e.g., reduce incidence of hypoglycemic events or hyperglycemic excursions), glucose control during and after meals (e.g., use historical information and trends to increase glycemic control), hyperglycemia corrections (e.g., increase time in target zone while avoiding hypoglycemic events from over-correction), and/or hypoglycemia treatments (e.g., address hypoglycemia while avoiding “rebound” hyperglycemia), to name a few.

Mobile health applications may provide such assistance in some form of guidance to the user. For example, guidance may include a graphical summary of a user's data over time or a mobile notification to the user, where the notification is offered to inform, warn, and/or recommend some action to the user. As an example, the application may help a user respond to a health condition in real time by predicting events or trends and provide treatment recommendations to address occurring or potential events or trends in real time. This type of calculated guidance and support may relieve the cognitive burden on the user. Some mobile health applications may also allow data to be exported in various formats to be shared with third parties and/or to connect users directly with healthcare professionals for feedback, which may support an improved patient-professional dialogue. Thus, if utilized, mobile health applications may increase quality of care while at the same time offering the potential to reduce costs for healthcare systems.

Health monitoring devices enable real-time sensing and analysis of human physiological information for medical monitoring of various diseases and conditions, noninvasive medical care and administration of various therapies and medicine, and mobile health and wellness monitoring. Portability is a central feature of these health monitoring devices. Thus, where continuously utilized, these devices may provide many benefits including improved information access, reduced medical errors, improved quality of care, etc.

To be an effective support tool, it may be desirable for the health monitoring device and/or application to continuously, or frequently, capture the attention of users and to stimulate the users' interest to actively engage with the device and/or application. Engagement indicates the degree of interaction a user has with the technology, e.g., device and/or application. Because health technologies, including health monitoring devices and mobile health applications, are voluntary use systems, the extent of user engagement with these technologies is generally determined by the users' perceived quality of experience, ongoing benefit of usage, and/or consideration of viable alternatives to using the technology.

As discussed herein, unfortunately, health monitoring systems, including health monitoring devices and/or mobile health applications, designed to support the management of chronic diseases or health conditions have been plagued with low user engagement and high user attrition rates. A reason for low user engagement and/or high user attrition rates may include failure of health monitoring systems to provide individualized or personalized decision support outputs (e.g., information, recommendations, warnings, etc.). When a mobile health application fails to provide individualized and/or personalized decision support outputs, users of the application may find the outputs to be ineffective in enabling them to take a holistic approach to managing their health (e.g., diseases, conditions, fitness, etc.). Further, decision support outputs that are not tailored to an individual (i.e., not “individualized”) may result in sub-optimal health outcomes. In addition, decision support outputs that are not tailored to a cohort that the individual is a member of (i.e., not “personalized”) may also result in sub-optimal health outcomes. As used herein, a “cohort” may be a set of “similar” users, with similar demographics, health history and/or other characteristics that influence outcomes. Thus, user engagement associated with such mobile health applications may decrease and, thereby, user attrition rates may increase.

As described above, a user (also referred to herein as a “patient”) may utilize one or more health monitoring devices, such as, but not limited to, one or more analyte sensors configured to report sensor outputs describing monitored physiological conditions of the user. The user may then use a software application, configured to execute on the user's display device, for receiving the sensor outputs. In certain embodiments, the application may utilize one or more decision support models to provide decision support outputs to the user based on the sensor outputs. For example, a decision support output may describe a recommended action for the user to perform (e.g., a recommended sleeping pattern for the user) or a recommended warning to the user about a predicted future physiological condition of the user (e.g., a warning that the user is likely to experience hyperglycemia in the next eight hours).

A decision support model may be a user-facing algorithm that determines a decision support output for a user based on the sensor outputs associated with the user. In some embodiments, the decision support model may include a scoring sub-model and a decision sub-model. The scoring sub-model may be configured to determine one or more risk scores for the user, where each risk score describes a predicted likelihood that the user experiences a particular condition (e.g., hyperglycemia or hypoglycemia) in a future time period (e.g., in the next eight hours). The scoring sub-model may be a trained machine learning model characterized by one or more trained parameters. The decision sub-model may be configured to select a decision support output for the user from a set of potential decision support outputs based on the risk scores for the user.

The decision sub-model may be characterized by one or more hyperparameters that define threshold values/conditions for the one or more risk scores. Each potential decision support output is associated with a respective subset of the threshold values/conditions that are defined by the hyperparameters of the decision sub-model. If the risk scores of a user satisfy the threshold values/conditions that are associated with a potential decision support output, then the decision sub-model selects the potential decision support output for providing to the user.

An example of a decision support model is a sleep advisor model that is configured to select a recommended sleep pattern for a user from a set of potential sleep patterns. In some embodiments, the scoring sub-model of the sleep advisor model is configured to determine at least one of a hyperglycemia risk score or a hypoglycemia risk score for the user based on sensor outputs associated with the user, where the hyperglycemia risk score describes a predicted likelihood that the user experiences hyperglycemia within a future time period (e.g., in the next eight hours) and the hypoglycemia risk score describes a predicted likelihood that the user experiences hypoglycemia in within a future time period.

The decision sub-model of the sleep advisor model is characterized by a set of hyperparameters that define two thresholds for each one of the potential sleep patterns: a hyperglycemia risk threshold and a hypoglycemia risk threshold. For example, the set of potential sleep patterns may include an 8-hour sleep pattern and a 10-hour sleep pattern. In this example, the 8-hour sleep pattern may be recommended to a user if the hyperglycemia risk score for the user satisfies a first threshold and the hypoglycemia risk score for the user satisfies a second threshold, while the 10-hour sleep pattern may be recommended to the user if the hyperglycemia risk score for the user satisfies a third threshold and the hypoglycemia risk score for the user satisfies a fourth threshold. These four threshold values may be defined by the hyperparameters of the decision sub-model of the sleep advisor model.

Another example of a decision support model is a hyperglycemia warning model that is configured to warn a user if the hyperglycemia warning model determines that the user is at the risk of hyperglycemia in a future time period. In this example, the scoring sub-model of the hyperglycemia warning model is configured to determine a hyperglycemia risk score for the user based on sensor outputs associated with the user, while the decision sub-model of the hyperglycemia warning model is configured to determine that a hyperglycemia warning should be presented to the user if the hyperglycemia risk score for the user satisfies (e.g., falls above) a hyperglycemia risk threshold.

In this example, the hyperglycemia risk threshold is an example of the sole hyperparameter of the decision sub-model, while the two potential decision support outputs of the hyperglycemia warning model include a “warning” output that is configured to cause a hyperglycemia warning to be presented to a user and a “no warning” output that is configured to prevent the hyperglycemia warning from being presented to the user. In another variation, the decision support output could be a pre-bedtime carb consumption (or exercise/insulin) recommendation, if risk of overnight hypoglycemia (or hyperglycemia) is predicted. These recommendations could have additional hyperparameters (on top of risk threshold) related to the type/amount of recommended carb consumption (or recommended exercise/insulin).

As the above examples illustrate, a decision support model may be associated with a set of trained parameters and a set of tunable/selectable hyperparameters. The above examples further illustrate that a decision support model may have any number of hyperparameters, and the number of hyperparameters of a decision support model may be a function of the number of risk scores that the scoring sub-model of the decision support model determines for a user and the number of potential decision support outputs that may be assigned to users by the decision sub-model of the decision support model.

Selecting user-specific hyperparameters (may also be referred to herein as “optimal hyperparameters”) for a decision support model is important for ensuring that the decision support outputs recommended by the model effectively respond to the needs of the users. Without optimal hyperparameters, a decision support output that is recommended by a decision support model fails to account for individualized or personalized needs of the user as determined based on the health journey of the user as well as demographic features of the user. As described above, providing ineffective decision support outputs to users in turn leads to higher user attrition rates from health-related applications that enable monitoring physiological conditions of the users to support better health condition management by the users. Accordingly many existing health-related applications suffer from high user attrition rates and low user engagement rates resulting from failure to provide individualized or personalized decision support outputs. In this context, individualization refers to determining decision support outputs by decision support models whose hyperparameters have been optimized for the conditions of an individual user, while personalization refers to providing decision support outputs by decision support models whose hyperparameters have been optimized for the conditions of a cohort of users (e.g., a cohort of users determined based on one or more demographic criteria, such as based on age).

Accordingly, the embodiments herein provide decision support models whose hyperparameters have been specifically determined for a user, thereby allowing for the decision support output to be specific to and effective for the user. In other words, given a set of users, the decision support model uses different sets of hyperparameters to determine the decision support outputs for the users, such that the decision support output for each user is determined in accordance with hyperparameters that are specifically assigned to a user and that may be different from hyperparameters assigned to other users. Determining and assigning user-specific hyperparameters to users may be performed using a multi-objective contextual multi-armed bandit (CMAB) algorithm, as described further below.

A multi-objective CMAB approach may provide various benefits including, but not limited to, allowing for a decision support model to optimize and assign hyperparameters for users as efficiently as possible. For example, a multi-objective CMAB approach avoids having to “test” many different values of hyperparameters for individual users to see what works best. Instead, the multi-objective CMAB approach involves training an algorithm to learn what the relationship is between user characteristics and optimal hyperparameter values that result in optimal outcomes for the user. In certain embodiments, the multi-objective CMAB algorithm assigns one or more hyperparameters to a user in a manner that is intended to both: (i) maximize the likelihood that decision support outputs generated based on the assigned hyperparameters lead to two or more outcomes/objectives of interest (e.g., a first outcome/objective of preventing the user from having glucose levels that are below an optimal glucose range and a second outcome/objective of preventing the user from having glucose levels that are above the optimal glucose range), and (ii) explore new values for hyperparameters while exploiting already-detected relationships between hyperparameters and the two or more outcomes/objectives of interest.

The systems, devices, and methods of the embodiments described herein can be used in conjunction with any type of analyte sensor for any measurable analyte. Further, the system, devices, and methods of the embodiment described herein may be used in conjunction with any health-related application that is provided to the user to improve the user's health. For example, a health-related application may help the user with treating a certain disease or just help with improving the health of a user who is not necessarily diagnosed with a disease.

### Example System with a Decision Support Engine for Determining Decision Support Outputs based on User-Specific Hyperparameters

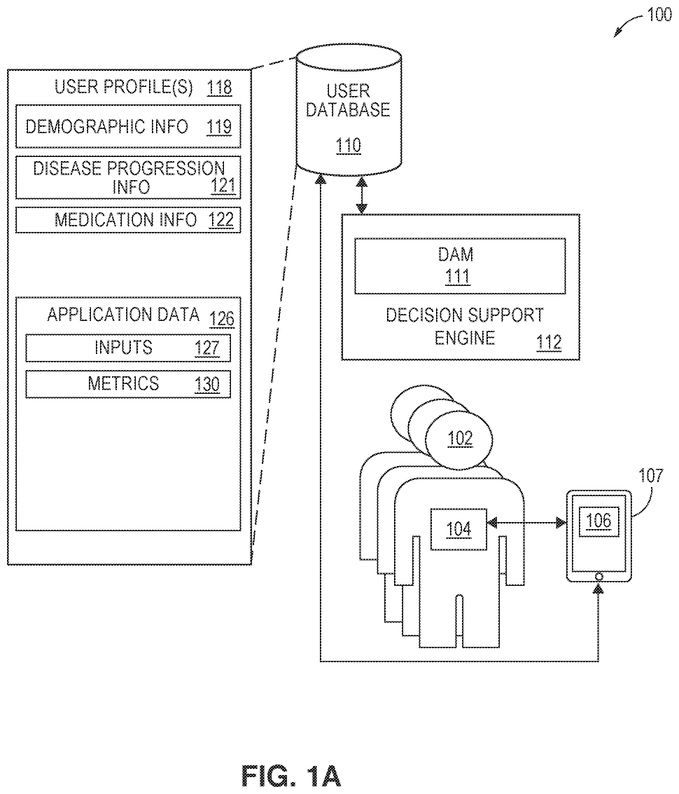

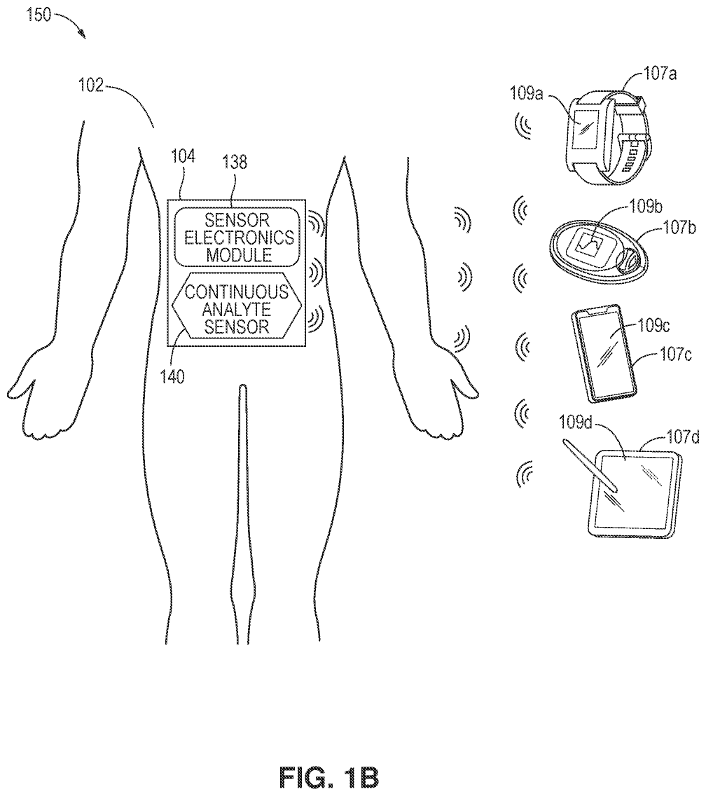

FIG. 1A illustrates an example health monitoring and support system in accordance with certain embodiments of the disclosure. The health monitoring and support system 100 may be utilized for monitoring user health, determining user-specific hyperparameters for decision support models, and providing decision support outputs to users associated with system 100. Each user of system 100, such as user 102, may interact with a mobile health application, such as mobile health application (“application”) 106 (e.g., a diabetes intervention application that provides decision support guidance), and/or a health monitoring device, such as an analyte monitoring system 104 (e.g., a glucose monitoring system). User 102, in certain embodiments, may be the patient or, in some cases, the patient's caregiver. In the embodiments described herein, the user is assumed to be the patient for simplicity only, but is not so limited. As shown, system 100 may include an analyte monitoring system 104, a mobile device 107 that executes application 106, a decision support engine 112 (including a Data Analysis Module (DAM) 111), and a user database 110.

Analyte monitoring system 104 may be configured to generate analyte measurements for the user 102, e.g., on a continuous basis, and transmit the analyte measurements to the mobile device 107 for use by application 106. In some embodiments, the analyte monitoring system 104 may transmit the analyte measurements to the mobile device 107 through a wireless connection (e.g., Bluetooth connection). In certain embodiments, mobile device 107 is a smart phone. However, in certain embodiments, mobile device 107 may instead be any other type of computing device such as a laptop computer, a smart watch, a tablet, or any other computing device capable of executing application 106.

Note that, while in certain examples the analyte monitoring system 104 is assumed to be a glucose monitoring system, analyte monitoring system 104 may operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates). Analytes can include naturally occurring substances, artificial substances, metabolites, and/or reaction products. In some embodiments, the analyte for measurement by the sensing regions, devices, and methods is albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, sodium, total protein, uric acid, metabolic markers, and drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

Application 106 may be a mobile health application that is configured to receive and analyze analyte measurements from the analyte monitoring system 104. In some embodiments, application 106 may transmit analyte measurements received from the analyte monitoring system 104 to a user database 110 (and/or the decision support engine 112), and the user database (and/or the decision support engine 112) may store the analyte measurements in a user profile 118 of user 102 for processing and analysis as well as for use by the decision support engine 112 to determine user-specific hyperparameters for decision support models and provide one or more decision support outputs such as, but not limited to, recommendations and/or guidance to the user 102 via the application 106. In some embodiments, application 106 may store the analyte measurements in a user profile 118 of user 102 locally for processing and analysis as well as for use by the decision support engine 112 to determine user-specific hyperparameters for decision support models and provide decision support outputs such as, but not limited to, recommendations and/or guidance to the user 102.

In certain embodiments, decision support engine 112 refers to a set of software instructions with one or more software modules, including a data analysis module (DAM) 111. In some embodiments, decision support engine 112 executes entirely on one or more computing devices in a private or a public cloud. In some other embodiments, decision support engine 112 executes partially on one or more local devices, such as mobile device 107, and partially on one or more computing devices in a private or a public cloud. In some other embodiments, decision support engine 112 executes entirely on one or more local devices, such as mobile device 107.

As discussed in more detail herein, decision support engine 112, may determine user-specific hyperparameters for decision support models and provide decision support outputs (e.g., decision support recommendations, etc.) to the user 102 via the application 106. For example, the decision support engine 112 may determine user-specific hyperparameters for decision support models by performing an initial exploration phase, a training phase, and an exploration-exploitation phase, as further described below. In some embodiments, the decision support engine 112 may determine user-specific hyperparameters for decision support models based on information including, but not limited to, information included in the user profile 118 stored in the user database 110. In some embodiments, the user profile 118 may include information collected about the user from the application 106, as further described below.

In certain embodiments, DAM 111 of decision support engine 112 may be configured to receive and/or process a set of inputs 127 (described in more detail below) (also referred to herein as “input data”) to determine one or more metrics 130 (also referred to herein as “metrics data”) that may then be used by decision support engine 112 in determining user-specific hyperparameters for decision support models. Inputs 127 may be stored in the user profile 118 in the user database 110. DAM 111 can fetch inputs 127 from the user database 110 and compute a plurality of metrics 130 which can then be stored as application data 126 in the user profile 118. Such metrics 130 may include health-related metrics.

In certain embodiments, application 106 is configured to take as input information relating to user 102 and store the information in a user profile 118 for user 102 in user database 110. For example, application 106 may obtain and record user 102′s demographic info 119, disease progression info 121, and/or medication info 122 in user profile 118. In certain embodiments, demographic info 119 may include one or more of the user's age, body mass index (BMI), ethnicity, gender, etc. In certain embodiments, disease progression info 121 may include information about the user 102's disease, such as, for diabetes, whether the user is Type I, Type II, pre-diabetes, or whether the user has gestational diabetes. In certain embodiments, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain embodiments, medication regimen info 122 may include information about the amount and type of medication taken by user 102, such as insulin or non-insulin diabetes medications and/or non-diabetes medication taken by user 102.

In certain embodiments, application 106 may obtain demographic info 119, disease progression info 121, and/or medication info 122 from the user 102 in the form of user input or from other sources. In certain embodiments, as some of this information changes, application 106 may receive updates from the user 102 or from other sources. In certain embodiments, user profile 118 associated with the user 102, as well as other user profiles associated with other users are stored in a user database 110, which is accessible to application 106, as well as to the decision support engine 112, over one or more networks (not shown). In certain embodiments, application 106 collects inputs 127 through user 102 input and/or a plurality of other sources, including analyte monitoring system 104, other applications running on mobile device 107, and/or one or more other sensors and devices. In certain embodiments, such sensors and devices include one or more of, but are not limited to, an insulin pump, other types of analyte sensors, sensors or devices provided by mobile device 107 (e.g., accelerometer, camera, global positioning system (GPS), heart rate monitor, etc.) or other user accessories (e.g., a smart watch), or any other sensors or devices that provide relevant information about the user 102. In certain embodiments, user profile 118 also stores application configuration information indicating the current configuration of application 106, including its features and settings.

User database 110, in some embodiments, refers to a storage server that may operate in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 may include other user profiles 118 associated with a plurality of other users served by health monitoring and decision support system 100. More particularly, similar to the operations performed with respect to the user 102, the operations performed with respect to these other users may utilize an analyte monitoring system, such as analyte monitoring system 104, and also interact with the same application 106, copies of which execute on the respective mobile devices of the other users 102. For such users, user profiles 118 are similarly created and stored in user database 110.

FIG. 1B illustrates a continuous analyte monitoring system in accordance with certain embodiments of the disclosure. The diagram 150 illustrates an example of an analyte monitoring system 104. In the example of FIG. 1B, the analyte monitoring system 104 is a glucose monitoring system. However, as described above, analyte monitoring system 104 may be configured for measuring any other analytes or a combination of multiple analytes. FIG. 1B illustrates a number of mobile devices 107a, 107b, 107c, and 107d (individually referred to as mobile device 107 and collectively referred to as mobile devices 107). Note that mobile device 107 of FIG. 1A may be any one of mobile devices 107a, 107b, 107c, or 107d. In other words, any one of mobile devices 107a, 107b, 107c, or 107d may be configured to execute application 106. The analyte monitoring system 104 may be communicatively coupled to mobile devices 107a, 107b, 107c, and/or 107d.

By way of an overview and an example, the analyte monitoring system 104 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data

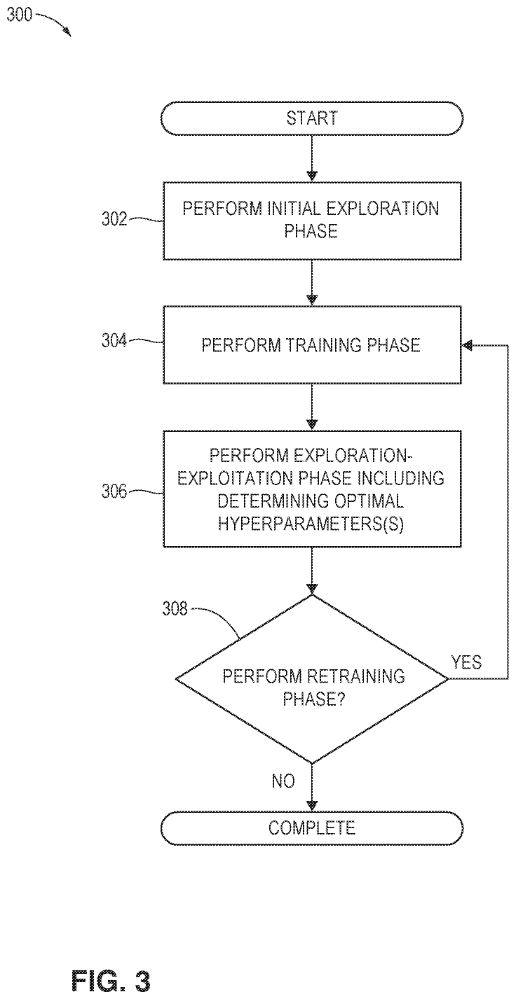

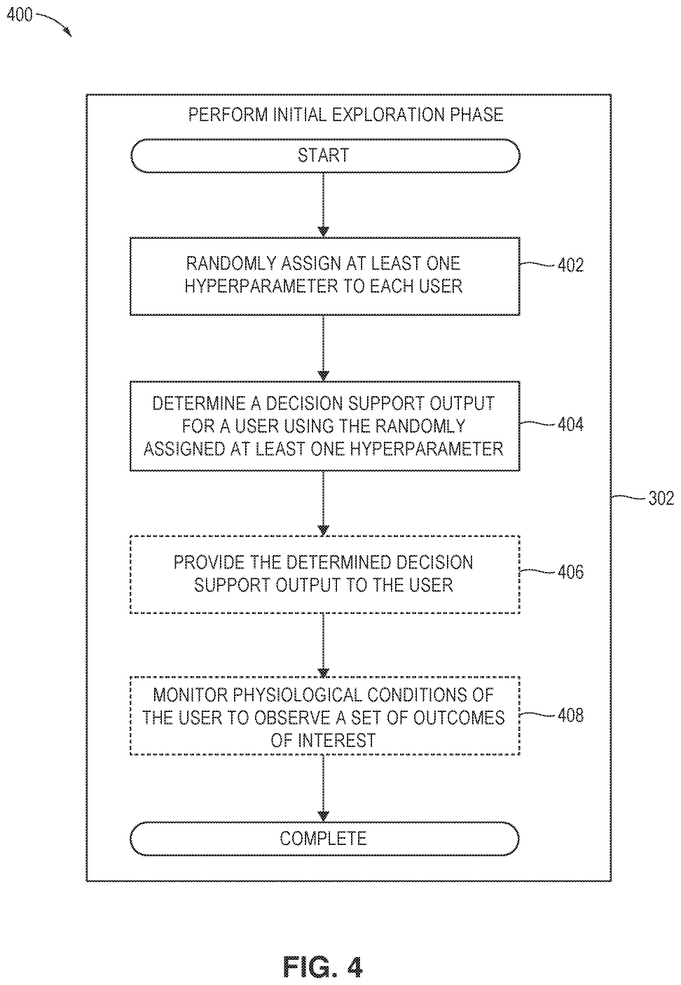

to remote devices, such as mobile devices 107. Paragraphs [0137]-[0140] and FIGS. 3A, 3B, and 4 of U.S. Patent Application Publication No. 2019/0336053 further describe an on-skin sensor assembly that, in certain embodiments, may be used in connection with analyte monitoring system 104. Paragraphs [0137]-[0140] and FIGS. 3A, 3B, and 4 of U.S. Patent Application Publication No. 2019/0336053 are incorporated herein by reference.

In certain embodiments, analyte monitoring system 104 includes an analyte sensor electronics module 138 and a continuous analyte sensor 140 (e.g., a glucose sensor) associated with the analyte sensor electronics module 138. In certain embodiments, analyte sensor electronics module 138 includes electronic circuitry associated with measuring and processing analyte sensor data (also referred to herein as “sensor outputs”) or information, including algorithms associated with processing and/or calibration of the analyte sensor data/information. Analyte sensor electronics module 138 may be physically/mechanically connected to the analyte sensor 140 and can be integral with (e.g., non-releasably attached to) or releasably attachable to the analyte sensor 140.

Analyte sensor electronics module 138 may also be electrically coupled to analyte sensor 140, such that the components may be electromechanically coupled to one another. Analyte sensor electronics module 138 may include hardware, firmware, and/or software that enable measurement and/or estimation of levels of the analyte in the user via analyte sensor 140 (e.g., which may be/include a glucose sensor). For example, analyte sensor electronics module 138 can include one or more potentiostats, a power source for providing power to analyte sensor 140, other components useful for signal processing and data storage, and a telemetry module for transmitting data from the sensor electronics module to various devices including, but not limited to, one or more display devices (e.g., the user's mobile device 107), user database 110, decision support engine 112, etc. Electronics can be affixed to a printed circuit board (PCB) within analyte monitoring system 104, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 138 may include sensor electronics that are configured to process sensor information, such as sensor data, and generate transformed sensor data and displayable sensor information. Examples of systems and methods for processing sensor analyte data are described in more detail herein and in U.S. Patent Nos. 7,310,544 and 6,931,327 and U.S. Patent Application Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

Analyte sensor 140 is configured to measure a concentration or level of the analyte in the user 102. The term analyte is further defined by paragraph [0117] of U.S. App. No. 2019/0336053. Paragraph [0117] of U.S. App. No. 2019/0336053 is incorporated herein by reference. In some embodiments, analyte sensor 140 comprises a continuous analyte sensor, such as a subcutaneous, transdermal (e.g., transcutaneous), or intravascular device. In some embodiments, analyte sensor 140 can analyze a plurality of intermittent blood samples. Analyte sensor 140 can use any method of analyte-measurement, including enzymatic, chemical, physical, electrochemical, spectrophotometric, polarimetric, calorimetric, iontophoretic, radiometric, immunochemical, and the like. Additional details relating to a continuous analyte sensor, such as a continuous glucose sensor, are provided in paragraphs [0072]-[0076] of U.S. Pat. No. 9,445,445. Paragraphs [0072]-[0076] of U.S. Pat. No. 9,445,445 are incorporated herein by reference.

With further reference to FIG. 1B, mobile devices 107 can be configured for displaying (and/or alarming) displayable sensor information that may be transmitted by sensor electronics module 138 (e.g., in a customized data package that is transmitted to the display devices based on their respective preferences). Each of mobile devices 107a, 107b, 107c, and/or 107d may respectively include a display such as touchscreen display 109a, 109b, 109c, and/or 109d for displaying a graphical user interface (e.g., of application 106) for presenting sensor information and/or analyte data to user 102 and/or receiving inputs from user 102. In certain embodiments, the mobile devices 107 may include other types of user interfaces such as voice user interface instead of or in addition to a touchscreen display for communicating sensor information to user 102 of the mobile device 107 and/or receiving user inputs. In certain embodiments, one, some, or all of mobile devices 107 may be configured to display or otherwise communicate the sensor information as it is communicated from sensor electronics module 138 (e.g., in a data package that is transmitted to respective display devices), without any additional prospective processing required for calibration and/or real-time display of the sensor data.

The mobile devices 107 may include a custom or proprietary display device, for example, analyte display device 107b, especially designed for displaying certain types of displayable sensor information associated with analyte data received from sensor electronics module 138 (e.g., a numerical value and/or an arrow, in certain embodiments). In certain embodiments, one of the mobile devices 107 includes a mobile phone, such as a smartphone that uses an Android, iOS, or another operating system configured to display a graphical representation of the continuous sensor data (e.g., including current and/or historic data).

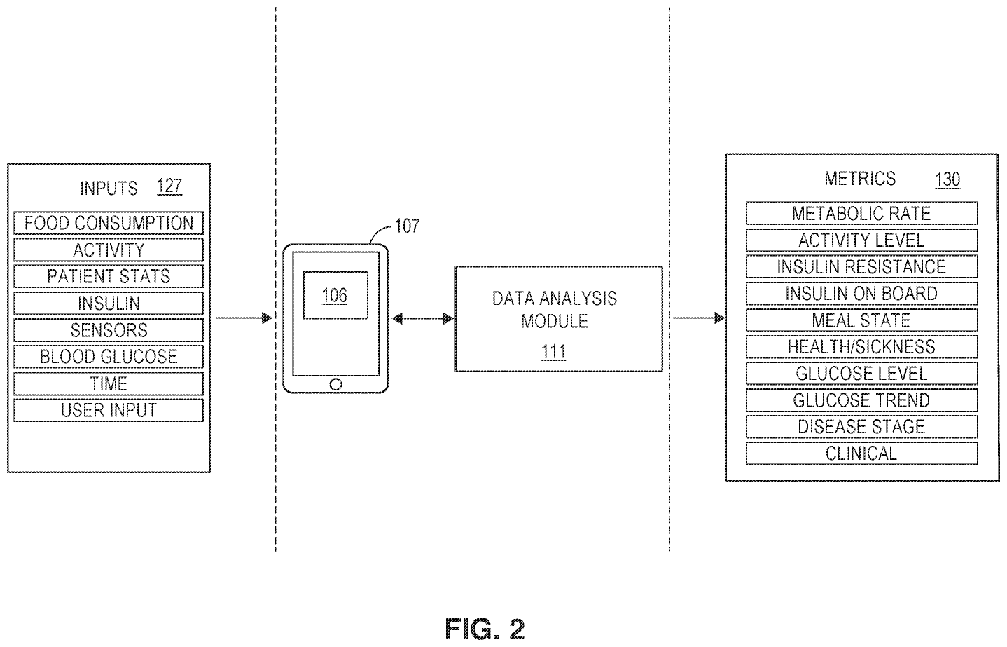

Example inputs and example metrics that are generated based on the inputs in accordance with certain embodiments of the disclosure are illustrated in FIG. 2. FIG. 2 illustrates example inputs 127 on the left, application 106 and DAM 111 in the middle, and example metrics 130 on the right. In certain embodiments, application 106 may obtain inputs 127 through one or more channels (e.g., manual user input, sensors, various applications executing on mobile device 107, etc.). Inputs 127 may be further processed by DAM 111 to output a plurality of metrics, such as metrics 130. In certain embodiments, inputs 127 and metrics 130 may also be used as contextual information by the DAM 111 and/or any computing device in the system 100 to perform various actions such as, but not limited to identifying a cohort and/or defining various sub-groups within a user cohort. Further, inputs (e.g., inputs 127) and metrics (e.g., metrics 130) may be used by the DAM 111 and/or any computing device in the system 100 to perform various processes in determining user-specific hyperparameters for decision support models, as further described below. Any of inputs 127 may be used for computing any of metrics 130. In certain embodiments, each one of metrics 130 may correspond to one or more values, e.g., discrete numerical values, ranges, or qualitative values (high/medium/low or stable/unstable).

In certain embodiments, inputs 127 include food consumption information. Food consumption information may include information about one or more of meals, snacks, and/or beverages, such as one or more of the size, content (carbohydrate, fat, protein, etc.), sequence of consumption, and time of consumption. In certain embodiments, food consumption may be provided by the user through manual entry, by providing a photograph through an application that is configured to recognize food types and quantities, and/or by scanning a bar code or menu. In various examples, meal size may be manually entered as one or more of calories, quantity (e.g., ‘three cookies’), menu items (e.g., ‘Royale with Cheese’), and/or food exchanges (1 fruit, 1 dairy). In some examples, meals may also be entered with the user's typical items or combinations for this time or context (e.g., workday breakfast at home, weekend brunch at restaurant). In some examples, meal information may be received via a convenient user interface provided by application 106.

In certain embodiments, inputs 127 include activity information. Activity information may be provided, for example, by an accelerometer sensor on a wearable device such as a watch, fitness tracker, and/or patch. In certain embodiments, activity information may also be provided through manual input by user 102.

In certain embodiments, inputs 127 include patient statistics, such as one or more of age, height, weight, body mass index, body composition (e.g., % body fat), stature, build, or other information. Patient statistics may be provided through a user interface, by interfacing with an electronic source such as an electronic medical record, and/or from measurement devices. The measurement devices may include one or more of a wireless, e.g., Bluetooth-enabled, weight scale and/or camera, which may, for example, communicate with the mobile device 107 to provide patient data.

In certain embodiments, inputs 127 include information relating to the user's insulin delivery. Such information may be received, via a wireless connection on a smart pen, via user input, and/or from an insulin pump. Insulin delivery information may include one or more of insulin volume, time of delivery, etc. Other configurations, such as insulin action time or duration of insulin action, may also be received as inputs.

In certain embodiments, inputs 127 include information received from sensors, such as physiologic sensors, which may detect one or more of heart rate, respiration, oxygen saturation, body temperature, etc. (e.g., to detect illness).

In certain embodiments, inputs 127 include glucose information. Such information may be provided as input, for example through analyte monitoring system 104. In certain embodiments, blood glucose information may be received from one or more of smart pill dispensers that track when the user takes medicine, a blood ketone meter, a laboratory-measured, or estimated A1C, other measures of long-term control, or sensors that measure peripheral neuropathy using tactile response, such as by using haptic features of a smartphone, or a specialty device.

In certain embodiments, inputs 127 include time, such as time of day, or time from a real-time clock.

As described above, in certain embodiments, DAM 111 determines or computes metrics 130 based on inputs 127 associated with user 102. An example list of metrics 130 is illustrated in FIG. 2. In certain embodiments, metrics 130 determined or computed by DAM 111 include metabolic rate. Metabolic rate is a metric that may indicate or include a basal metabolic rate (e.g., energy consumed at rest) and/or an active metabolism, e.g., energy consumed by activity, such as exercise or exertion. In some examples, basal metabolic rate and active metabolism may be tracked as separate metric. In certain embodiments, the metabolic rate may be calculated by DAM 111 based on one or more of inputs 127, such as one or more of activity information, sensor input, time, user input, etc.

In certain embodiments, metrics 130 determined or computed by DAM 111 include an activity level metric. The activity level metric may indicate a level of activity of the user. In certain embodiments, the activity level metric be determined, for example based on input from an activity sensor or other physiologic sensors. In certain embodiments, the activity level metric may be calculated by DAM 111 based on one or more of inputs 210, such as one or more of activity information, sensor input, time, user input, etc.

In certain embodiments, metrics 130 determined or computed by DAM 111 include an insulin sensitive metric (also referred to herein as an “insulin resistance”). The insulin sensitivity metric may be determined using historical data, real-time data, or a combination thereof, and may, for example, be based upon one or more inputs 127, such as one or more of food consumption information, blood glucose information, insulin delivery information, the resulting glucose levels, etc. In certain embodiments, the insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, metrics 130 determined or computed by DAM 111 include a meal state metric. The meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, metrics 130 determined or computed by DAM 111 include health and sickness metrics. Health and sickness metrics may be determined, for example, based on one or more of user input (e.g., pregnancy information or known sickness information), from physiologic sensors (e.g., temperature), activity sensors, or a combination thereof. In certain embodiments, based on the values of the health and sickness metrics, for example, the user's state may be defined as being one or more of healthy, ill, rested, or exhausted.

In certain embodiments, metrics 130 determined or computed by DAM 111 include glucose level metrics. Glucose level metrics may be determined from sensor information (e.g., blood glucose information obtained from analyte monitoring system 104). In some examples, a glucose level metric may also be determined, for example, based upon historical information about glucose levels in particular situations, e.g., given a combination of food consumption, insulin, and/or activity. In certain embodiments, a blood glucose trend may be determined based on the glucose level over a certain period of time.

In certain embodiments, metrics 130 determined or computed by DAM 111 include a disease stage. For example disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, metrics 130 determined or computed by DAM 111 include clinical metrics. Clinical metrics generally indicate a clinical state a user is in with respect to one or more conditions of the user, such as diabetes. For example, in the case of diabetes, clinical metrics may be determined based on glycemic measurements, including one or more of A1C, trends in A1C, time in range, time spent below a threshold level, time spent above a threshold level, and/or other metrics derived from blood glucose values. In certain embodiments, clinical metrics may also include one or more of estimated A1C, glycemic variability, hypoglycemia, and/or health indicator (time magnitude out of target zone).

As discussed herein, DAM 111 and/or application 106 may be implemented in one or more computing devices to perform various processes for determining user-specific hyperparameters for decision support models. For example, such processes may include performing (1) an initial exploration phase, (2) a training phase, and (3) an exploration-exploitation phase, as further described below. For example, an initial exploration phase may be performed by assigning a random set of hyperparameters, determining and providing decision support outputs, and monitoring physiological conditions by analyzing monitoring data. In certain embodiments, the monitoring data may include any data that provides information about physiological conditions of the user including, but not limited to, the inputs 127 and other metrics 130.

As further described below, a training phase may be performed by applying training data to an outcome prediction model to determine predicted outcomes. In some embodiments, the training data may include input data of a user (e.g., contextual data, inputs 127, etc.), input data of a hyperparameter (e.g., a value associated with the hyperparameter), and/or monitoring data. In certain embodiments, contextual data may be personal (based on cohort data) or individual (based on specific user data).

In addition, an exploration-exploitation phase made be performed by dividing a set of users into an exploration subset and an exploitation subset of users. In certain embodiments, the exploration-exploitation phase may also include determining decision support outputs for the exploitation subset of users using the experimental set of hyperparameters, determining one or more predicted outcomes, and determining one or more scalarized outcomes. In various embodiments, the exploration-exploitation phase may also include selecting and assigning the experimental set of hyperparameters having an optimal scalarized outcomes, and determining decision support outputs using an optimal set of hyperparameters. Further, the computing device may monitor the physiological conditions of the user to observe the set of outcomes of interest (e.g., a time above optimal glucose range and a time below optimal glucose range) that occur after the decision support output is provided to the user and/or after the user performs actions recommended by the decision support output. In certain embodiments, the (2) training phase (herein also referred to as “retraining phase”) and the (3) exploration-exploitation phases may be repeated to further improve the user-specific hyperparameters for decision support models. In such embodiments, the retaining phase may be performed using data from the previous exploration subset of users, as further described below.

### Example Processes for Determining Decision Support Outputs based on User-Specific Hyperparameters

FIG. 3 is a flow diagram illustrating a process 300 for determining decision support outputs based on user-specific hyperparameters in accordance with certain embodiments of the disclosure. In various embodiments, the user-specific hyperparameters may include a set of hyperparameters for each user in a set of users. The user-specific hyperparameters may be determined using a multi-objective CMAB algorithm executed by a computing device. The computing device may be part of the health monitoring and support system 100, such a computing device that executes decision support engine 112 or a backend server (not shown).

In reference to FIG. 3, the process 300 may include performing (block 302) an initial exploration phase, as further described below. During an initial exploration phase, the computing device assigns a random set of hyperparameters to each user, determine a decision support output using the random hyperparameter set, and provide the decision support output to the user. The initial exploration phase may also include monitoring physiological conditions to observe one or more outcomes of interest (e.g., a time above optimal glucose range and a time below optimal glucose range) that occur after the decision support output is provided to the user or after the user performs actions recommended by the decision support output.

Further, the process 300 may include performing (block 304) a training phase, as further described below. During the training phase, the computing device uses monitoring data from the initial exploration phase to train an outcome prediction model (also referred to herein as a “tactic assignment algorithm”). The outcome prediction model is a machine learning model that is configured to determine a set of predicted outcomes for a specific user and a respective set of hyperparameters. Each predicted outcome is an outcome that is predicted to occur if a decision support output, that is determined based on the respective set of hyperparameters, is provided to the user and/or if the user performs the action recommended by the decision support output. One example of an outcome predicted for a user and respective set of hyperparameters include a time duration that the user is predicted to be above an optimal glucose range if a decision support output, that is determined using the respective set of hyperparameters, is provided to the user. Another example of a predicted outcome is a time duration that the user is predicted to be below the optimal glucose range if the decision support output, that is determined using the respective set of hyperparameters, is provided to the user.

In addition, the process 300 may also include performing (block 306) an exploration-exploitation phase. During an exploration-exploitation phase, the computing device divides the set of users into an exploitation subset and an exploration subset. With respect to users in the exploitation subset, the computing device uses predicted outcome values determined by the outcome prediction model to assign optimal sets of hyperparameters to those users. With respect to users in the exploration subset, the computing device continues to randomly assign hyperparameters to those users.

After assigning hyperparameters to each user, the computing device uses the assigned hyperparameters of each user to determine the decision support output for the user based on a decision support model. Further, the computing device provides the determined decision support output to the user and monitors the physiological conditions of the user to observe the set of outcomes of interest (e.g., a time above optimal glucose range and a time below optimal glucose range) that occur after the decision support output is provided to the user and/or after the user performs actions recommended by the decision support output.

The process 300 may also include determining whether to perform a retraining phase (block 308), for example, based on the resulting monitoring data. Determining whether to perform a retraining phase may include reviewing the monitoring data to see if the user's metrics (e.g., health/sickness, glucose level, glucose trend, disease stage, etc.) have improved or deteriorated. For example, if the user has been provided decision support outputs based on the user-specific hyperparameters, but the monitoring data indicates that the user's metrics have deteriorated, then the process 300 may decide to perform a retaining phase (block 308). In another example, if the user has been provided decision support outputs based on the user-specific hyperparameters, but the monitoring data indicates that the user's metrics have not improved or have not improved enough, then the process 300 may decide to perform a retaining phase (block 308). In some embodiments, the process 300 may determine to retrain periodically (e.g. every month, regardless of performance). In some embodiments, the process 300 may determine to retrain periodically every time the sample size of exploration set of users' hyperparameters and observed outcomes hits a threshold number (e.g. 10,000). In some embodiments, the process 300 may determine to retrain if it is observed that, for exploitation subset of users, the predicted outcomes for the optimal hyperparameters, have started deviating more than a threshold amount from the subsequently observed outcomes.

If the process 300 determines (block 308) to perform the retraining phase, then the process 300 may repeating blocks 304 and 306. In a subsequent iteration of block 304, the computing device proceeds to retrain (block 304) the outcome prediction machine learning model using the data (e.g., randomly assigned hyperparameter(s), user context, and outcomes observed) from the exploration subset of users. Moreover, in the subsequent iteration of block 306, the computing device performs (block 306) the exploration-exploitation phase using the retrained outcome prediction model including determining and providing new decision support outputs to the users.

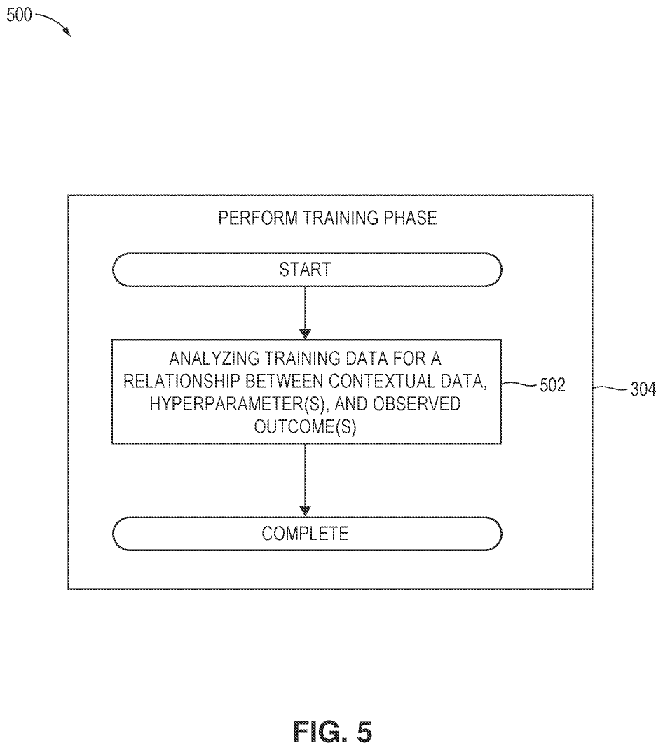

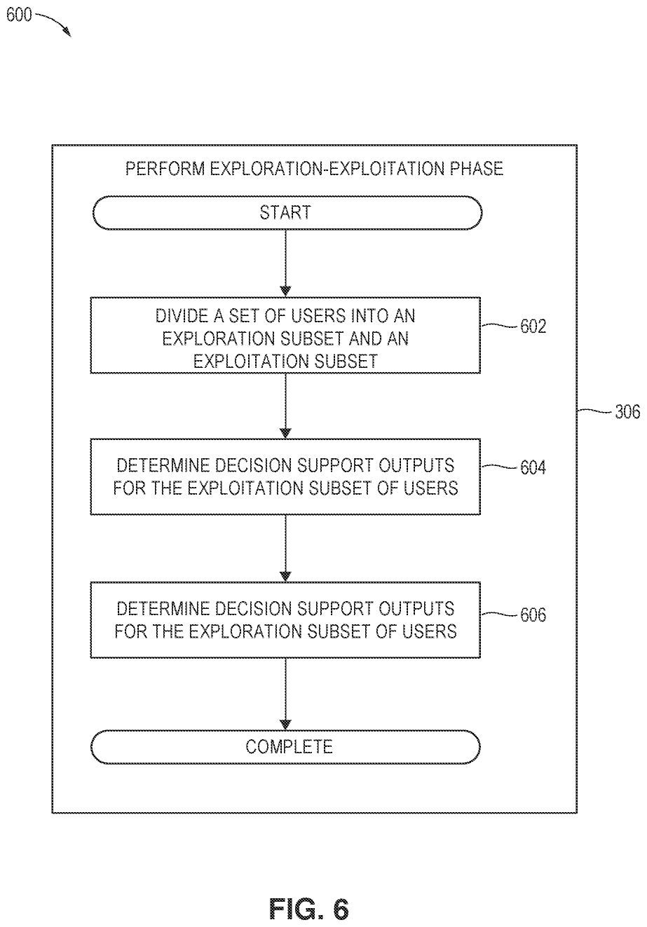

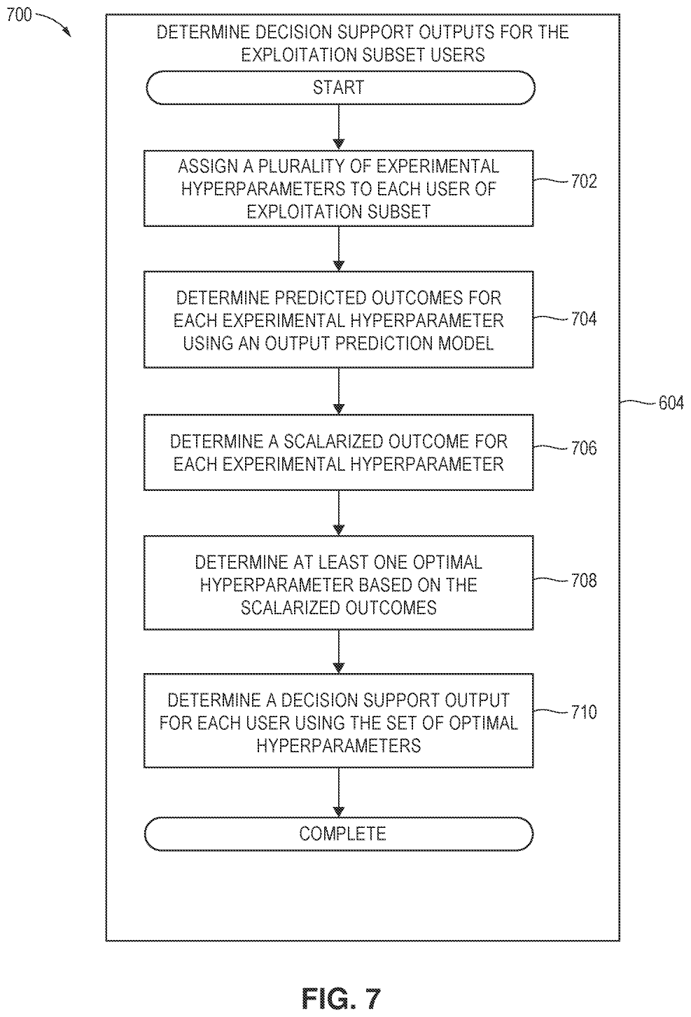

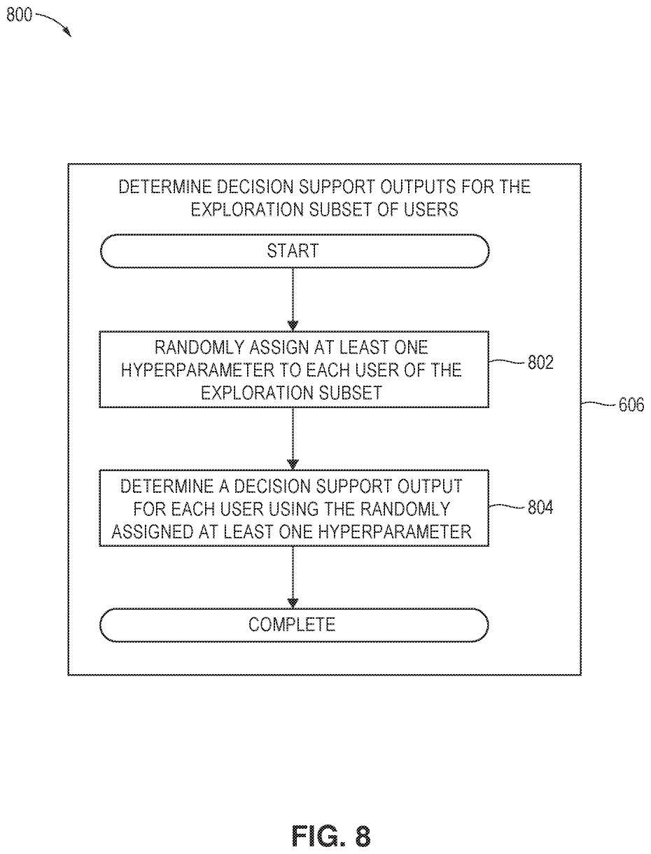

Below, blocks 302, 304, 306 are described in more detail and by reference to subsequent FIGS. 4-8. In particular, block 302 of FIG. 3 is described in more detail by reference to blocks 402-408 of FIG. 4. Further, block 304 of FIG. 3 is described in more detail by reference to block 502 of FIG. 5. Furthermore, block 306 of FIG. 3 is described in more detail by reference to blocks 602-606 of FIG. 6. Then block 604 of FIG. 6 is described in more detail by reference to blocks 702-710 of FIG. 7. After block 604, block 606 of FIG. 6 is then described in more detail by reference to blocks 802 and 804 of FIG. 8.

### 1. Block 302: Performing the Initial Exploration Phase

As described above in reference to FIG. 3, process 300 may include performing (block 302) an initial exploration phase. FIG. 4 is a flow diagram illustrating a process 400 for performing (block 302) the initial exploration phase of FIG. 3, in accordance with certain embodiments of the disclosure. The process 400 includes randomly assigning (block 402) one or more hyperparameters to each user. For example, the computing device randomly assigns a set of hyperparameters to each user. In some embodiments, to randomly assign a hyperparameter to a user, the computing device randomly selects a value from an acceptable range for the hyperparameter. In some embodiments, to randomly assign a hyperparameter to a user, the computing device first determines a cohort-specific range for the hyperparameter based on a cohort of the user and then randomly selects a value from the cohort-specific range. For example, the computing device may first determine that the hyperglycemia risk threshold for females between 20 to 30 years of age should be between 0.2-0.7, and then randomly selects a value for a user that is a female having 25 years of age from the range (0.2, 0.7).

In reference to FIG. 4, the process 400 may include determining (block 404) one or more decision support outputs for a user using the at least one randomly assigned hyperparameter. For example, after the computing device randomly assigns a hyperparameter set to a user, the computing device uses the randomly assigned hyperparameter set to determine a decision support output for the user. As an example, the computing device may randomly assign a hyperglycemia risk threshold of 0.5 to a user. The computing device may use the hyperglycemia risk threshold as the hyperparameter of the decision sub-model of a hyperglycemia warning model to determine whether to warn the user that she is at the risk of hyperglycemia in a future time period.

In some embodiments, the process 400 may include providing (block 406) the determined decision support output to the user. In the above example, if the hyperglycemia risk score determined by the scoring sub-model of the hyperglycemia warning model for the user is, for example, 0.6, then the decision sub-model may determine that a hyperglycemia warning should be communicated to this user because 0.6 is greater than the threshold value of 0.5. In certain embodiments, the process 400 may include monitoring (block 408) physiological conditions of the user to observe a set of outcomes of interest. For example, the computing device may provide a hyperglycemia warning and then monitor for the user's glucose levels. As another example, the computing device may provide a recommendation to the user to exercise, drink water, etc. and then monitor the user's glucose levels either after providing the recommendation(s) and/or after the user has implemented the recommendation(s).

### 2. Block 304: Performing the Training Phase

As described above in reference to FIG. 3, process 300 may include performing (block 304) a training phase. FIG. 5 is a flow diagram illustrating a process 500 for performing (block 304) a training phase in accordance with certain embodiments of the disclosure. The training phase may be performed to initially train an outcome prediction model or to retrain an already-trained outcome prediction model based on new monitoring data determined by monitoring physiological conditions of the user. The outcome prediction model (also referred to here as a “tactic assignment algorithm”) is a machine learning model that is configured to determine one or more predicted outcomes (e.g., a set of predicted outcomes) for a respective user if a decision support output that is determined by a decision support model in accordance with a respective set of hyperparameters is provided to the respective user and/or if the respective user performs actions recommended by the decision support output.

In reference to FIG. 5, the process 500 may include analyzing (block 502) training data for a relationship between users' contextual data, hyperparameters, and observed outcomes (e.g., monitoring data). For example, the output prediction model may receive training data such as, but not limited to, hyperparameter (e.g., the randomly assigned hyperparameter(s)), contextual data (e.g., user data such as age, demographic data, health history, etc.), and observed outcomes (e.g., monitoring data). In some embodiments, input data to each execution of the outcome prediction model (e.g., training data) may include the input data of the user (e.g., contextual data of the respective user) and the input data of the hyperparameters (e.g., a value of the respective set of hyperparameter (herein also referred to as “hyperparameter value”)). In some embodiments, input data to each execution of the outcome prediction model may also include the monitoring data (e.g., one or more outcomes associated with one or more randomly assigned hyperparameter).

In certain embodiments, the training data may also include a set of training data entries, where each training data entry includes contextual data associated with a respective user, data associated with the respective hyperparameter(s), and a set of monitored/observed outcomes (e.g., monitoring data) that resulted after a decision support output determined using the respective hyperparameter was provided to the user and/or after the user performed actions recommended by the described decision support output. In various embodiments, the training data for training the outcome prediction model may result from monitoring physiological conditions of users after decision support outputs are provided to the users, where the decision support outputs provided to the users may be determined (in initial training contexts) using operations of the initial exploration phase or (in retraining contexts) using operations of the exploration-exploitation phase where the retraining utilizes data from the exploration subset of users.

### 3. Block 306: Performing the Exploration-Exploitation Phase

As described above in reference to FIG. 3, process 300 may include performing (block 306) an exploration-exploitation phase. In certain embodiments, the exploration-exploitation phase may be performed using a multi-object CMAB. During the exploration-exploitation phase, a set of users may be divided into an exploration subset and an exploitation subset and decision support outputs may be determined for both subsets of users.

FIG. 6 is a flow diagram illustrating a process 600 for performing (block 306) an exploration-exploitation phase in accordance with certain embodiments of the disclosure. The process 600 may include dividing (block 602) a set of users into an exploration subset and an exploitation subset. For example, the set of users are first randomly divided into an exploration subset and an exploitation subset in accordance with an exploration ratio ε. In particular, a ε fraction of the users are randomly selected and used to identify the exploration subset, and the remaining (1−ε) fraction of the users are used to identify an exploitation subset. In some embodiments, the magnitude of the exploration ratio ε decreases after each retraining of the outcome prediction model. In some embodiments, the magnitude of the exploration ratio ε is set to zero after a predefined number of retrainings of the outcome prediction model. In certain embodiments, the magnitude of the decrease in exploration ratio ε may be a function of the outcomes model accuracy (which may improve over time). In addition, the exploration ratio ε may also have a lower bound>0, to ensure some degree of exploration is always performed since what works best for users may change over time.

During the exploration-exploitation phase, the multi-object CMAB model may also determine decision support outputs for the exploitation subset of users and the exploration subset of users. Various example processes for determining decision support outputs for the exploration and the exploitation subset of users are further described below by reference to FIG. 6. In particular, block 604 of FIG. 6 describes determining decision support outputs for the exploitation subset of users while block 606 of FIG. 6 describes determining decision support outputs for the exploration subset of users.

a. Block 604: Determining the Decision Support Outputs for the Exploitation Subset

The process 600 for performing (block 306) an exploration-exploitation phase may also include determining (block 604) decision support outputs for the exploitation subset of users. Various techniques can be used to determine (block 604) decision support outputs for the exploitation subset of users. For example, after assigning a user to the exploitation subset, the following operations may be performed to determine the decision support output for the exploitation subset user.

FIG. 7 is a flow diagram illustrating a process 700 for determining (block 604) decision support outputs for the exploitation subset of users in accordance with certain embodiments of the disclosure. The process 700 may include assigning (block 702) a plurality of experimental hyperparameters (e.g., two or more sets of experimental hyperparameters) to each user of the exploitation subset. In some embodiments, every potential hyperparameter set that may be assigned to the decision support model is experimentally assigned to every user in the exploitation subset. For example, if the decision support model is characterized by only one hyperparameter that can take one of A potential values, then all of the A potential values are experimentally assigned to the user. As another example, if the decision support model is characterized by two hyperparameters that can take B and C potential values, respectively, then all of the B*C pairings of the potential values for the two hyperparameters are experimentally assigned to the user. In some embodiments (e.g., when at least one hyperparameter has a continuous range), a subset of all potential hyperparameter sets are randomly assigned to the user in the exploitation subset. For example, if a decision support model is characterized by a hyperparameter that can take a value from a continuous range, then a defined number of randomly-selected values from the continuous range may be experimentally assigned to the user. In a further example for a hyperparameter with a continuous range, intervals may be defined to select the continuous hyperparameter. For example, if the accepted values are [0.2 to 0.8], the interval could be 0.01 and the experimentally assigned values could be [0.20, 0.21, 0.22 . . . 0.79, 0.80]. Accordingly, at the end of this step, multiple sets of experimental hyperparameters are assigned to each user in the exploitation subset.

In reference to FIG. 7, the process 700 may include determining (block 704) predicted outcomes (e.g., a set of predicted outcomes) for each experimental hyperparameter (e.g., for each set of experimental hyperparameters) using an output prediction model. For example, after the sets of experimental hyperparameters are assigned to the user, the outcome prediction model can be used to determine a set of predicted outcomes for the user with respect to each set of experimental hyperparameters. In certain embodiments, the input data for the user (e.g., contextual data) and the input data for each one of the sets of experimental hyperparameters (e.g., values associated the experimental hyperparameters) is provided to the outcome prediction model to determine a set of predicted outcomes for the user with respect to the set of experimental hyperparameters. For example, given a user who is a female, 25 years of age, and has a hyperglycemia risk threshold of 0.43 that is experimentally assigned to the user, the outcome prediction model may process contextual data (e.g., age data, gender data, location data, and/or health history data) associated with the user and the hyperglycemia risk threshold of 0.43 to determine: (i) a predicted time that the user will be above an optimal glucose range if a decision support output that is determined using the hyperglycemia risk threshold of 0.43 is provided to the user, and (ii) a predicted time that the user will be below the optimal glucose range if the decision support output that is determined using the hyperglycemia risk threshold of 0.43 is provided to the user. Accordingly, at the end of this step, a set of (e.g., two or more) predicted outcomes are determined for each set of experimental hyperparameters assigned to the user.

In certain embodiments, the output prediction model may determine one or more predicted outcomes specific to the user and respective hyperparameter(s). For example, during each execution, the outcome prediction model outputs a set of predicted outcomes that are specific to both a respective user and a respective set of hyperparameters. As described further above, the training data including, but not limited to, contextual data of the respective user, the respective set of hyperparameters (e.g., values associated with the hyperparameter(s)), and observed outcomes (e.g., monitoring data) may be used as inputs to train the outcome prediction model. Output data resulting from each execution of the outcome prediction model includes a one or more predicted outcomes (e.g., a set of predicted outcomes), such as the combination of a predicted time above an optimal glucose range and a predicted time below the optimal glucose range. For example, in an exemplary execution, the outcome prediction model processes contextual data of a respective user and a respective set of hyperparameters for a decision support model to determine: (i) a predicted time above an optimal glucose range if a decision support output that is generated by a decision support model based on the respective set of hyperparameters is provided to the respective user and/or if the respective user performs actions recommended by the described decision support output, and (ii) a predicted time above the optimal glucose range if the described decision support output is provided to the respective user and/or if the respective user performs actions recommended by the described decision support output.

In further reference to FIG. 7, the process 700 may also include determining (block 706) a scalarized outcome for each experimental hyperparameter (e.g., for each set of experimental hyperparameters) by combining the predicted outcomes (e.g., the set of predicted outcomes). In certain embodiments, after determining a set of predicted outcomes for each set of experimental hyperparameters, the set of predicted outcomes for the set of experimental hyperparameters are combined to determine a scalarized outcome for the set of experimental hyperparameters. In some embodiments, the set of predicted outcomes for a set of experimental hyperparameters include two or more predicted outcomes, such as a predicted time duration that the user will be above an optimal glucose range if a decision support output that is determined using the respective set of hyperparameters is provided to the user and a predicted time duration that the user will be below the optimal glucose range if the decision support output is provided to the user.

In some embodiments, the set of predicted outcomes for a set of experimental hyperparameters are combined in accordance with a set of predefined outcome weights to generate the scalarized outcome for the set of experimental hyperparameters. For example, the scalarized outcome for a hyperglycemia risk threshold of 0.43 may be determined based on the result of w₁*o₁+w₂*o₂, where: (i) o₁ is the predicted time duration that the user will be above the optimal glucose range if a decision support output that is determined using the hyperglycemia risk threshold of 0.43 is provided to the user, (ii) w₁ is the outcome weight of o₁, (iii) o₂ is the predicted time duration that the user will be below the optimal glucose range if that decision support output that is determined using the hyperglycemia risk threshold of 0.43 is provided to the user, (ii) w₂ is the outcome weight of o₂. In a further example, avoiding time below range may be more important than avoiding time above range. In this example, the CMAB model may set w₁ and w₂ to, for example, 0.2 and 0.8, respectively. In another example, avoiding time below range may be equally important as avoiding time above range. In this example, the CMAB model may set w₁=w₂=0.5. Accordingly, at the end of this step, each set of experimental hyperparameter sets is associated with a single scalarized outcome.

In further reference to FIG. 7, the process 700 may also include determining (block 708) at least one optimal hyperparameter based on the scalarized outcomes. In some embodiments, the computing device may select and assign to each user a set of experimental hyperparameters having an optimal scalarized outcome. In certain embodiments, after determining a scalarized outcome for each set of experimental hyperparameters, the set of experimental hyperparameters having the optimal (e.g., lowest) scalarized outcome is selected and assigned to the user. For example, if the set of experimental hyperparameters include a hyperglycemia risk threshold of 0.43 that is associated with a scalarized outcome of 25 and a hyperglycemia risk threshold of 0.66 that is associated with a scalarized outcome of 20, then the hyperglycemia risk threshold of 0.66 may be assigned to the user to minimize the predicted time outside of optimal glucose range when the CMAB model sets w₁=w₂=0.5. For any other choice of weights, the CMAB model minimizes the scalarized time outside the optimal glucose range (potentially weighting time above or below range more heavily). Accordingly, at the end of this step, the exploitation subset user is assigned a single set of optimal hyperparameters.

In further reference to FIG. 7, the process 700 may also include determining (block 710) a decision support output for each user using the at least one optimal hyperparameter (e.g., the set of optimal hyperparameters). For example, after assigning a set of optimal hyperparameters to the user, the set of optimal hyperparameters are used by the decision sub-model of the decision support model to determine the decision support output for the user. For example, if the set of optimal hyperparameters for the user include the hyperglycemia risk threshold of 0.66, and if the scoring sub-model of the decision support model has generated a score of 0.33 for the user, then because 0.33 falls below 0.66, the computing device may determine a “no warning” output that corresponds to not providing a hyperglycemia warning to the user.

b. Block 606: Determining Decision Support Outputs for the Exploration Subset

The process 600 for performing (block 306) the exploration-exploitation phase may also include determining (block 606) decision support outputs for the exploration subset of users (as illustrated in FIG. 8). Various techniques can be used to determine decision support outputs for the exploration subset of users. For example, after assigning a user to the exploration subset, the following operations are performed to determine the decision support output for the exploration subset user.

FIG. 8 is a flow diagram illustrating a process 800 for determining (block 606) decision support outputs for the exploration subset of users in accordance with certain embodiments of the disclosure. The process 800 may include randomly assigning (block 802) at least one hyperparameter to each user of the exploration subset of users. For example, a randomly-selected set of hyperparameters is assigned to the user. In some embodiments, to assign a random hyperparameter to a user, the computing device first determines a cohort-specific range for the hyperparameter based on a cohort of the user and then randomly selects a value from the cohort-specific range. For example, the computing device may first determine that the hyperglycemia risk threshold for females between 20 to 30 years of age should be between 0.2-0.7, and then randomly select a value for a user that is a female having 25 years of age from the range (0.2, 0.7).

In further reference to FIG. 8, the process 800 may include determining (block 804) a decision support output for each user in the exploration subset using the randomly assigned at least one parameter. For example, after randomly assigning a set of hyperparameters to the user, the set of random hyperparameters are used by the decision sub-model of the decision support model to determine the decision support output for the user. For example, if the set of random hyperparameters for the user includes the hyperglycemia risk threshold of 0.23, and if the scoring sub-model of the decision support model has generated a score of 0.33 for the user, then because 0.33 does not fall below 0.23, the computing device may determine a “warning” output that corresponds to providing a hyperglycemia warning to the user.

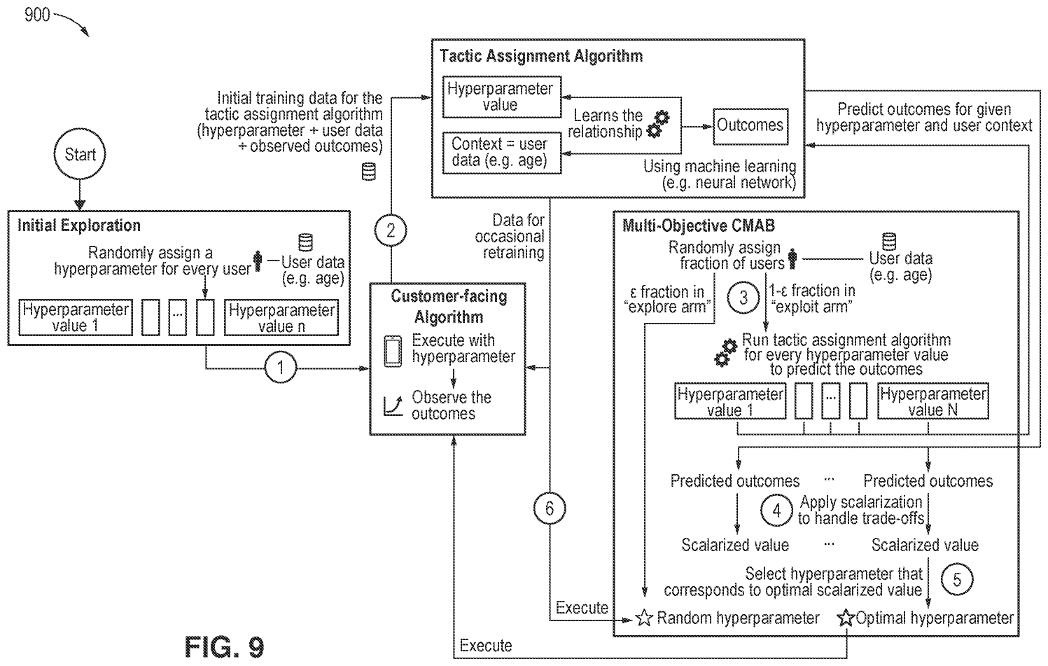

Although specific categorization of steps for an initial exploration phase, a training phase, and an exploration-exploitation phase are described above with respect to FIGS. 3-8, various other categorizations of steps may be utilized in accordance with embodiments of the present disclosure. For example, as illustrated in FIG. 9 and further described below, processes for determining user-specific hyperparameters for use with decision support models may be categorized as steps and not in any particular phase. Further, although specific steps are described as being performed by specific algorithms above with respect to FIGS. 3-8, various other algorithms may be utilized to perform various steps in accordance with embodiments of the present disclosure. For example, in some embodiments, determining and/or providing decision support outputs based on a hyperparameter and monitoring outcomes may be performed by a customer-facing algorithm executing decision support models.

### Exemplary Operational Flow Diagram for Determining Decision Support Outputs based on User-Specific Hyperparameters

As describe above, customer-facing algorithms may execute decision support models. For example, a decision support model may include determining a decision support output utilizing user-specific hyperparameters. In some embodiments, executing a decision support model may include determining a decision support output for a user by using: (i) an optimal level range for the user 102 as input data, and/or (ii) a risk tolerance profile based on hyperparameters including user-specific hyperparameters.

FIG. 9 is a flow diagram illustrating an exemplary operational process for determining decision support outputs based on user-specific hyperparameters in accordance with certain embodiments of the disclosure. Specifically, a nocturnal hypoglycemia classifier with a tunable probability threshold hyperparameter, and Time below Range (TbR) and Time above Range (TaR) as outcomes of interest further described below in reference to FIG. 9.

The process 900 may include assigning (Step 1) a random set of hyperparameters to each user. For example, during the initial exploration phase, the CMAB model may test the nocturnal hypoglycemia classifier by measuring TbR and TaR for a range of K different probability thresholds (0.1, 0.2, . . . , 0.9). Users are randomly assigned one of these probability thresholds. In certain embodiments, a customer-facing algorithm may execute the decision support model using the randomly assigned probability thresholds (i.e., random set of hyperparameter) and observer the outcomes, as further described above.

The process 900 may also include determining (Step 2) decision support outputs based on the assigned set of hyperparameters, observing outcomes associated with the decision support outputs, and training an outcome prediction model based on the observed data. For example, the process may train the tactic assignment algorithm on this exploration phase data, to learn the relationship between parameter (probability threshold), context (e.g. age and diabetes type), and observed outcome(s) of interest (TbR and TaR).

In reference to FIG. 9, the process 900 may include dividing (Step 3) the users into an exploitation subset and an exploitation subset using a ε fraction, as further described above. For example, for the 1−ε fraction of users that get assigned to the exploit arm of the MAB, the process may run the tactic assignment algorithm K times (with probability thresholds 0.1, 0.2, . . . , 0.9) for each user to predict their TbR and TaR.

In addition, the process 900 may include determining (Step 4) scaled outcomes for users in the exploitation subset by scaling predicted outcomes determined using the outcome prediction model. For example, for each predicted (TbR, TaR) for each user, the process may include applying scalarization (e.g., 0.5*TbR+0.5*TaR) to deal with a trade-off between TbR and TaR. In this example, scalarization weights of 0.5 for TbR and TaR are used. Alternatively, the process could determine weights through user input (e.g., users who value no overnight hypoglycemia over anything else, people who generally don't care because they will be woken up by an alarm, etc.). In another alternative, the process could determine weights by cohorting the users either by stated preference or change the metric to be weighted (e.g., User_Risk_Tolerance*TbR+(1−User_Risk_Tolerance)*TaR). In a further alternative, the process could set the weights as default value that users can change themselves.

In further reference to FIG. 9, the process 900 may include determining (Step 5) optimal hyperparameter sets for the users in exploitation subset based on the scaled outcomes and assigning the optimal hyperparameters sets to those users. For example, for each user in the exploitation subset of users, the process may pick the probability threshold that maximizes 0.5*TbR+0.5*TaR and use that threshold to run the nocturnal hypoglycemia classifier for that patient. Moreover, the process 900 may also include randomly assigning (Step 6) hyperparameters to users in the exploration subset and using this data and observed outcomes to retrain an outcome prediction model. For example, the process may keep assigning a ε fraction of users randomly to the exploration subset of users, and periodically use this data to retrain the tactic assignment algorithm. In certain embodiments, the process 900 may repeat Steps 2-6.

### Example Apparatus for Determining Decision Support Outputs based on User-Specific Hyperparameters

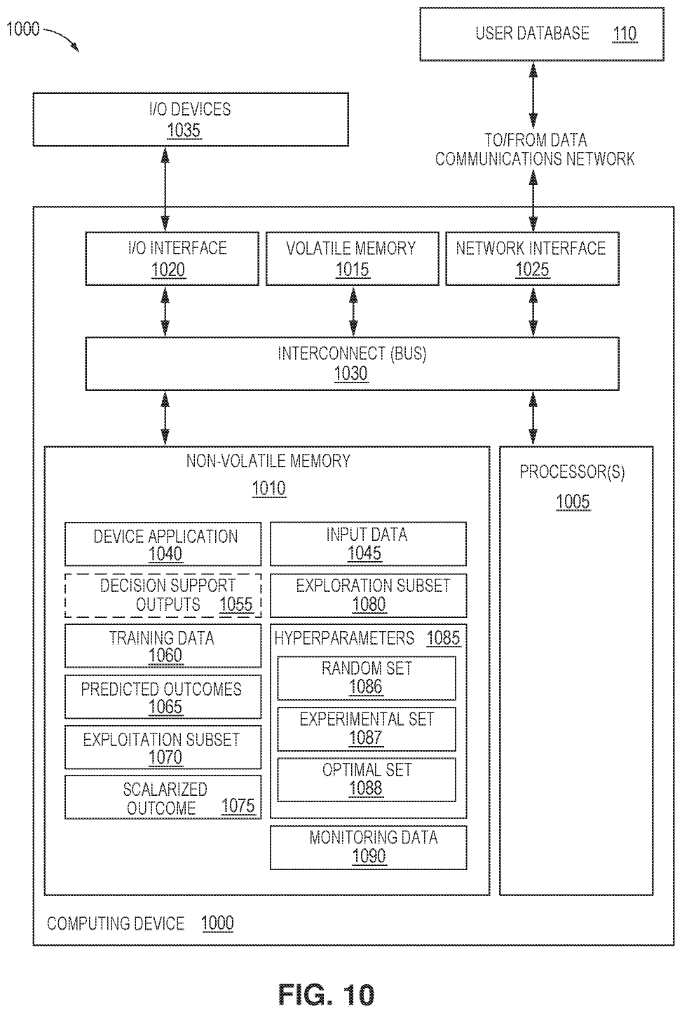

FIG. 10 is a block diagram depicting a computing device 1000 configured to determine user-specific hyperparameters for decision support models, according to certain embodiments disclosed herein. Although depicted as a single physical device, in embodiments, computing device 1000 may be implemented using virtual device(s), and/or across a number of devices, such as in a cloud environment. Computing device 1000 may be display device 107, a server, multiple serves, or any combination thereof.

As illustrated, computing device 1000 includes a one or more processor(s) 1005, non-volatile memory 1010, volatile memory 1015, a network interface 1025, and one or more input/output (I/O) interfaces 1020. In the illustrated embodiment, processor 1005 retrieves and executes programming instructions stored in the non-volatile memory 1010 and/or the volatile memory 1015, as well as stores and retrieves data residing in the non-volatile memory 1010 and/or the volatile memory 1015. In certain embodiments, non-volatile memory 1010 is configured to store instructions (e.g., computer-executable code, device application 1040) that when executed by processor(s) 1005, cause processor(s) 1005 to perform the processes and/or operations described herein and illustrated in FIGS. 3-9. In certain embodiments, non-volatile memory 1010 stores code for executing the functions of the DAM 111, decision support engine 112, and/or the application 106. Note that computing device 1000 may be configured to perform the functions of only one of the DAM 111, decision support engine 112, and/or the application 106, in which case additional system(s) may be used for performing the functions of the others.

Processor(s) 1005 is generally representative of a single central processing unit (CPU) and/or graphics processing unit (GPU), multiple CPUs and/or GPUs, a single CPU and/or GPU having multiple processing cores, and the like. Volatile memory 1015 is generally included to be representative of a random access memory (RAM). Non-volatile memory 1010 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1035 (such as keyboards, monitors, etc.) can be connected via the I/O interface(s) 1020. Further, via network interface 1025, computing device 1000 can be communicatively coupled with one or more other devices and components, such as user database 110. In certain embodiments, computing device 1000 is communicatively coupled with other devices via a network, which may include the Internet, local network(s), and the like. The network may include wired connections, wireless connections, or a combination of wired and wireless connections. As illustrated, processor(s) 1005, non-volatile memory 1010, volatile memory 1015, network interface 1025, and I/O interface(s) 1020 are communicatively coupled by one or more bus interconnects 1030. In certain embodiments, computing device 1000 is a server executing in an on-premises data center or a cloud environment. In certain embodiments, the computing device 1000 is a user's mobile device.

In the illustrated embodiment, the non-volatile memory 1010 may include a device application 1040 that configures the processor(s) 1005 to perform various processes and/or operations in determining user-specific hyperparameters 1085 for decision support models, as described above. In some embodiments, the device application 1040 may perform the functions of the DAM 111, the decision support engine 112, and/or the application 106. As described above in reference to FIGS. 3-9, the computing device 1000 may be configured to perform an initial exploration phase by assigning a random set 1086 of hyperparameters, determining and providing a decision support outputs 1055, and monitoring physiological conditions by receiving and/or analyzing monitoring data 1090 (e.g., inputs 127, metrics 130, etc.), as further described above. In addition, the computing device 1000 may be configured to perform a training phase by receiving and/or inputting training data 1060 to an outcome prediction model and determining, using the outcome prediction model, predicted outcomes 1065, as further described above. In some embodiments, the training data 1060 may include input data of a user (e.g., contextual data), input data of a hyperparameter (e.g., a value associated with the hyperparameter), and/or monitoring data 1090. Moreover, the computing device 1000 may be configured to perform an exploration-exploitation phase by dividing a set of users into an exploration subset 1080 and an exploitation subset 1070. In certain embodiments, the exploration-exploitation phase may also include determining decision support outputs 1055 for the exploitation subset of users using an experimental set 1087 of hyperparameters, determining predicted outcomes 1065, and determining a scalarized outcome 1075, as further described above. In various embodiments, the exploration-exploitation phase may also include selecting and assigning the experimental set 1087 of hyperparameters having an optimal scalarized outcomes, and determining decision support outputs 1055 using an optimal set 1088 of hyperparameters, as further described above. In certain embodiments, the computing device 1000 may also receive and/or store input data 1045 (e.g., inputs 127). Furthermore, the computing device 1000 may be configured to receive and/or generate monitoring data (e.g., inputs 127, metrics 130, etc.), as described above.

Further, although specific operations (e.g., operations of FIG. 3-9) and data are described as being performed and/or stored by a specific computing device above with respect to FIG. 10, in certain embodiments, a combination of computing devices may be utilized instead.

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” In this document, the term “set” or “a set of” a particular item is used to refer to one or more than one of the particular item.

Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel,” “perpendicular,” “round,” or “square,” are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A non-transitory computer readable storage medium storing a program comprising instructions that, when executed by at least one processor of a computing device, cause the at least one processor to perform operations including:
performing an initial exploration phase by:
randomly assigning at least one hyperparameter to each user of a plurality of users;
performing a training phase by:
analyzing training data, wherein the training data comprises contextual data and a value associated with the at least one randomly assigned hyperparameter; and
determining a relationship between the contextual data and the value associated with the at least one randomly assigned hyperparameter; and
performing an exploration-exploitation phase by:
dividing the plurality of users into an exploration subset of users and an exploitation subset of users;
determining at least one optimal hyperparameter for each user of the exploitation subset of users;
determining, using the at least one optimal hyperparameter, at least one decision support output for each user of the exploitation subset of users;
randomly assigning at least one hyperparameter to each user of the exploration subset of users; and
determining, using the at least one randomly assigned hyperparameter, at least one decision support output for each user of the exploration subset of users.

2. The non-transitory computer readable storage medium of claim 1, wherein the initial exploration phase is further performed by:
determining, using the at least one randomly assigned hyperparameter, a decision support output for each user of the plurality of users;
providing the decision support output to each user of the plurality of users; and
receiving monitoring data for each user of the plurality of users, wherein the monitoring data provides information to at least one physiological condition.

3. The non-transitory computer readable storage medium of claim 2, wherein the training data further comprises the monitoring data, and the training phase is further performed by determining a relationship between the monitoring data, the contextual data, and the value associated with the at least one randomly assigned hyperparameter.

4. The non-transitory computer readable storage medium of claim 1, wherein the at least one optimal hyperparameter for each user of the exploitation subset of users is determined by:
assigning a plurality of experimental hyperparameters to each user of the exploitation subset;
determining at least one predicted outcome for each experimental hyperparameter of the plurality of experimental hyperparameters;
determining a scalarized outcome for each experimental hyperparameter of the plurality of experimental hyperparameters; and
determining the at least one optimal hyperparameter based on the scalarized outcome.

5. The non-transitory computer readable storage medium of claim 4, wherein the operations further comprise:
performing a retraining phase by:
analyzing training data, wherein the training data comprises contextual data, a value associated with the randomly assigned hyperparameter of the exploration subset of users, and monitoring data for the exploration subset of users; and
determining a relationship between the contextual data, the value associated with the at least one randomly assigned hyperparameter of the exploration subset of users, and the monitoring data for the exploration subset of users.

6. The non-transitory computer readable storage medium of claim 1, wherein the exploration-exploitation phase is performed using a contextual multi-armed bandit algorithm.

7. The non-transitory computer readable storage medium of claim 4, wherein the at least one predicted outcome is determined using a tactic assignment algorithm.

8. A method for determining user-specific hyperparameters for decision support models, the method comprising:
performing an initial exploration phase by:
randomly assigning at least one hyperparameter to each user of a plurality of users;
performing a training phase by:
analyzing training data, wherein the training data comprises contextual data and a value associated with the at least one randomly assigned hyperparameter; and
determining a relationship between the contextual data and the value associated with the at least one randomly assigned hyperparameter; and
performing an exploration-exploitation phase by:
dividing the plurality of users into an exploration subset of users and an exploitation subset of users;
determining at least one optimal hyperparameter for each user of the exploitation subset of users;
determining, using the at least one optimal hyperparameter, at least one decision support output for each user of the exploitation subset of users;
randomly assigning at least one hyperparameter to each user of the exploration subset of users; and
determining, using the at least one randomly assigned hyperparameter, at least one decision support output for each user of the exploration subset of users.

9. The method of claim 8, wherein the initial exploration phase is further performed by:
determining, using the at least one randomly assigned hyperparameter, a decision support output for each user of the plurality of users;
providing the decision support output to each user of the plurality of users; and
receiving monitoring data for each user of the plurality of users, wherein the monitoring data provides information to at least one physiological condition.

10. The method of claim 9, wherein the training data further comprises the monitoring data, and the training phase is further performed by determining a relationship between the monitoring data, the contextual data, and the value associated with the at least one randomly assigned hyperparameter.

11. The method of claim 8, wherein the at least one optimal hyperparameter for each user of the exploitation subset of users is determined by:
assigning a plurality of experimental hyperparameters to each user of the exploitation subset;
determining at least one predicted outcome for each experimental hyperparameter of the plurality of experimental hyperparameters;
determining a scalarized outcome for each experimental hyperparameter of the plurality of experimental hyperparameters; and
determining the at least one optimal hyperparameter based on the scalarized outcome.

12. The method of claim 11 further comprising:
performing a retraining phase by:
analyzing training data, wherein the training data comprises contextual data, a value associated with the randomly assigned hyperparameter of the exploration subset of users, and monitoring data for the exploration subset of users; and
determining a relationship between the contextual data, the value associated with the at least one randomly assigned hyperparameter of the exploration subset of users, and the monitoring data for the exploration subset of users.

13. The method of claim 8, wherein the exploration-exploitation phase is performed using a contextual multi-armed bandit algorithm.

14. The method of claim 11, wherein the at least one predicted outcome is determined using a tactic assignment algorithm.

15. A computing device for determining user-specific hyperparameters for decision support models, the computing device comprising:
a network interface;
a processor operatively connected to the network interface;
a memory storing a program comprising instructions that, when executed by the processor, cause the computing device to:
perform an initial exploration phase by:
randomly assigning at least one hyperparameter to each user of a plurality of users;
perform a training phase by:
analyzing training data, wherein the training data comprises contextual data and a value associated with the at least one randomly assigned hyperparameter; and
determining a relationship between the contextual data and the value associated with the at least one randomly assigned hyperparameter; and
perform an exploration-exploitation phase by:
dividing the plurality of users into an exploration subset of users and an exploitation subset of users;
determining at least one optimal hyperparameter for each user of the exploitation subset of users;
determining, using the at least one optimal hyperparameter, at least one decision support output for each user of the exploitation subset of users;
randomly assigning at least one hyperparameter to each user of the exploration subset of users; and
determining, using the at least one randomly assigned hyperparameter, at least one decision support output for each user of the exploration subset of users.

16. The computing device of claim 15, wherein the initial exploration phase is further performed by:
determining, using the at least one randomly assigned hyperparameter, a decision support output for each user of the plurality of users using a decision support model;
providing the decision support output to each user of the plurality of users; and
receiving monitoring data for each user of the plurality of users, wherein the monitoring data provides information to at least one physiological condition.

17. The computing device of claim 16, wherein the training data further comprises the monitoring data, and the training phase is further performed by determining a relationship between the monitoring data, the contextual data, and the value associated with the at least one randomly assigned hyperparameter.

18. The computing device of claim 15, wherein the at least one optimal hyperparameter for each user of the exploitation subset of users is determined by:
assigning a plurality of experimental hyperparameters to each user of the exploitation subset;
determining a predicted outcome for each experimental hyperparameter of the plurality of experimental hyperparameters;
determining a scalarized outcome for each experimental hyperparameter of the plurality of experimental hyperparameters; and
determining the at least one optimal hyperparameter based on the scalarized outcome.

19. The computing device of claim 15, wherein the exploration-exploitation phase is performed using a contextual multi-armed bandit algorithm.

20. The computing device of claim 18, wherein the computing device is further configured to:
perform a retraining phase by:
analyzing training data, wherein the training data comprises contextual data, a value associated with the randomly assigned hyperparameter of the exploration subset of users, and monitoring data for the exploration subset of users; and
determining at least one predicted outcome for each user of the exploration subset of users based on a relationship between the contextual data, the value associated with the at least one randomly assigned hyperparameter of the exploration subset of users, and the monitoring data for the exploration subset of users.
